(19) United States
(12) Patent Application Publication
(10) Pub. No.: US 2019/0060107 A1
(43) Pub. Date: Feb. 28, 2019

(54) Convective Swaddle Blanket

(71) Applicant: 3M INNOVATIVE PROPERTIES COMPANY, St. Paul, MN (US)

(72) Inventors: Matthew Zabel, Lake Elmo, MN (US); Michael Vostal, Minneapolis, MN (US); Poeuth Pann, Woodbury, MN (US); Jared J. Balthazor, Linwood TWP, MN (US); Melanie L. Collins, Minneapolis, MN (US); Philip G. Dion, Blaine, MN (US); Daniel P. Doran, Minneapolis, MN (US); Marc A. Egeland, Minneapolis, MN (US); Yvonne Lund, Roberts, WI (US); Glenn R. Maharaj, Minneapolis, MN (US); Andrew J. McGregor, West Lakeland, MN (US); John Stark, St. Paul, MN (US)

(21) Appl. No.: 16/110,269

(22) Filed: Aug. 23, 2018

Related U.S. Application Data
(60) Provisional application No. 62/549,224, filed on Aug. 23, 2017.
(60) Provisional application No. 62/549,302, filed on Aug. 23, 2017.

(51) Int. Cl. A61F 7/00; A61F 7/02

(52) U.S. Cl. CPC A61F 7/0097; A61F 7/0085; A61F 7/02; A61F 2007/0258; A61F 2007/0091; A61F 2007/0226; A61F 2007/0231; A61F 2007/006

(57) ABSTRACT

Aspects of the present disclosure relate to a convective blanket that includes an air permeable upper sheet having a periphery. The convective blanket includes a lower sheet bonded to the upper sheet with a peripheral seal proximate to the periphery of the upper sheet to form an interior space therein between the upper and lower sheets. The convective blanket includes a wing portion partially surrounded by a portion of the peripheral seal and a body portion. Additional aspects relate to a system including the convective blanket and a method that uses the convective blanket.

### BACKGROUND

Warming a person during surgery affords clinical benefits, such as prevention or treatment of hypothermia, encouragement of immune system function, promotion of wound healing, reduction of infection rates, and mitigation of discomfort. An inflatable blanket laid over a person's body is used to warm the person perioperatively. Such a blanket, when inflated with heated air, warms the person principally by convection of warmed air through a permeable portion of the blanket, although conduction and radiation of heat from the blanket also contribute to warming.

However, covering a person for warming may interfere with a variety of surgical procedures by preventing or limiting access to the person. For example, during cardiac surgery access to the thorax, groin and extremities is necessary when blood vessels must be harvested from the legs and arms for bypass construction on the heart. Covering a person with a convective warming blanket may interfere with, if not prevent, the necessary access. Adapting the blanket to avoid interference by reducing its size may compromise its capacity to warm the person. Adapting the blanket to provide access by separable seals may extend and complicate the surgery due to need to integrate the operation and management of the blanket with surgical protocols.

One way to achieve the benefits of warming a person during clinical procedures while providing unobstructed access to the person is to deploy an inflatable underbody convective warming blanket (“underbody blanket” or “blanket”) beneath the person. The person is laid on the underbody blanket which warms the person by convection of warmed air, and by conduction and radiation, without covering the person. Examples of underbody blankets include an inflatable convective pad described in the assignee's U.S. Pat. No. 8,177,828, the inflatable pad described in the assignee's U.S. Pat. No. 6,102,936, and the assignee's underbody series blankets illustrated and described at www.3m.com.

A preferred warming blanket construction includes permeable and impermeable sheets that are sealed together at their peripheries to form an interior space therein between the sheets. Patterns of interior seals between the sheets within the peripheral seal form air distribution structures in the interior space that are intended to maintain a uniform temperature within the warming blanket with a minimal loss of heat. One or more inlet ports are provided in the construction to admit warmed air into the interior space. The warmed air pressurizes and inflates the blanket, and the inflation pressure forces warmed air through the permeable sheet which faces the person when the blanket covers the person. The inlet ports are typically provided in the impermeable sheet, within the peripheral seal.

Warming blankets are not generally constructed to be used as underbody devices. Rotation of the warming blanket such that the blanket is underneath the person places the impermeable sheet and the inlet port against a supporting surface such as a surgical table, which makes it difficult to couple an air hose to the port. Further, the pressure of the person's body against the inlet port can impede or, in some cases, pinch off the air distribution structures and thereby block distribution of warmed air in the warming blanket. Thus, the supporting device must be modified to accommodate an air hose, and/or the warming blanket must be placed so as to locate the inlet port beyond the periphery of the supporting device.

While underbody blankets can be useful during surgical procedures, portions of the underbody blanket may not be dimensioned as to cover the sides or anterior portions of the person. Further, underbody blankets may be difficult to use during pre-warming procedures.

### SUMMARY

Aspects of the present disclosure relate to a convective blanket that includes an air permeable upper sheet having a periphery. The convective blanket includes a lower sheet bonded to the upper sheet with a peripheral seal proximate to the periphery of the upper sheet to form an interior space therein between the upper and lower sheets. The convective blanket includes a wing portion partially surrounded by a portion of the peripheral seal and a body portion.

Additional aspects relate to a system including the convective blanket and a method that uses the convective blanket.

### BRIEF DESCRIPTION OF DRAWINGS

FIG. 1A illustrates a plan view of an embodiment of a convective blanket, according to aspects of the present disclosure.

FIG. 1B illustrates a plan view of the embodiment in FIG. 1 A from the bottom side, according to aspects of the present disclosure.

FIG. 1C illustrates a side cross-sectional view of the embodiment in FIG. 1A taken from the line 1-1, according to aspects of the present disclosure.

FIG. 1D illustrates a side cross-sectional view of the embodiment in FIG. 1A taken from the line 2-2, according to aspects of the present disclosure.

FIG. 2A illustrates a plan view of an embodiment of a convective blanket from an upper sheet, according to aspects of the present disclosure.

FIG. 2B illustrates a plan view of an embodiment of a convective blanket from a lower sheet, according to aspects of the present disclosure.

FIG. 3A illustrates a plan view of an embodiment of the convective blanket in FIG. 2A, according to aspects of the present disclosure.

FIG. 3B illustrates a plan view of the convective blanket in FIG. 3A except folded over a patient in a swaddle position, according to aspects of the present disclosure.

FIG. 4 illustrates a plan view of an embodiment of an interwoven convective blanket, according to aspects of the present disclosure.

FIG. 5 illustrates a plan view of an embodiment of a convective blanket with a plurality of ducts, according to aspects of the present disclosure.

### DETAILED DESCRIPTION

Aspects of the present disclosure relate to an underbody blanket with a wing portion such that the wing portion “wraps over” onto the anterior portion of the patient (particularly over the abdominal cavity). The underbody blanket can be used to facilitate pre-warming before surgical procedures. In at least one embodiment of the present disclosure, a wing portion can be fluidically isolated from the remainder of the blanket such that only the peripheral portions of a patient are warmed which can be particularly advantageous during pre-warming of a patient. An underbody convective warming blanket has two ends, upper and lower sides, and interior space with an air distribution (i.e., inflatable) structure. At least one inlet port opens through the underbody blanket, preferably through an edge thereof, into the interior space. Preferably, the underbody blanket is inflatable. That is to say, the underbody blanket, flaccid when not in use, tautens when a stream of pressurized air is received in the interior space by way of an inlet port.

The underbody blanket has an upper surface on which a person may lie. At least a portion or portions of the upper surface is/are permeable to air. In a typical or preferred deployment, the underbody blanket is laid, or rests, on a supporting surface with the lower side on the supporting surface. When a stream of warmed pressurized air is received in the interior space, warmed air circulates in the air distribution structure, through the permeable upper surface, to warm a person lying on the upper side. In at least one embodiment, the lower side of the underbody blanket can be coated with a layer of low-friction material such as polytetrafluorethylene or other low friction plastics.

A preferred embodiment of an underbody convective warming blanket shown in the figures and described in this specification may be constructed using techniques and materials which are known in the art, or which are equivalent thereto. Generally, the construction and materials with which an underbody convective blanket may be made include two or more sheets of flexible material that are brought together in a manufacturing process. For example, the material may be a spunbound non-woven synthetic material, one side of which is extrusion coated (or lined) with a heat and/or glue sealable plastic. The material for the sheet forming the upper side may be processed by formation of apertures through to configure the sheet's permeability.

The sheets are brought together with the plastic linings facing each other and bonded, joined, or sealed by heat, glue, welding, or any equivalent, at a periphery by a peripheral seal, and at multiple points within the peripheral seal. For example, the sealing process can use various techniques, such as ultrasonic welding, radio frequency welding, heat sealing, or the like. In at least one embodiment, the seal can also be formed through adhesive bonding or mechanical fastening. The seals described herein can be formed as continuous air impervious seals (such as those found in the periphery) or discontinuous air permeable seals. Other materials and/or alternative construction methods may also be employed. One exemplary construction for the underbody convective warming blanket is described in US Publication No. 2006/0052851 Al. The peripheral seal joins the two or more sheets to form an interior space between the sheets. One sheet (the “upper sheet”) forms the upper side of the underbody blanket; the other sheet (the “lower sheet”) forms the lower side of the underbody blanket.

The upper sheet has at least one permeable surface portion through which warmed, pressurized air circulates, passes, or exits to warm a person lying on the upper side. One or more inlet ports, each with provision for retention of an air hose nozzle, are provided through the underbody blanket for admitting a stream of warmed, pressurized air into the interior space, from an air hose connected to a heater/blower unit. Warmed air circulating through the one or more permeable surface portions causes the person to be warmed. Although convection of the warmed air is the principal mode of warming, the underbody convective warming blanket itself, when provided with warmed air, may also warm by radiation and conduction.

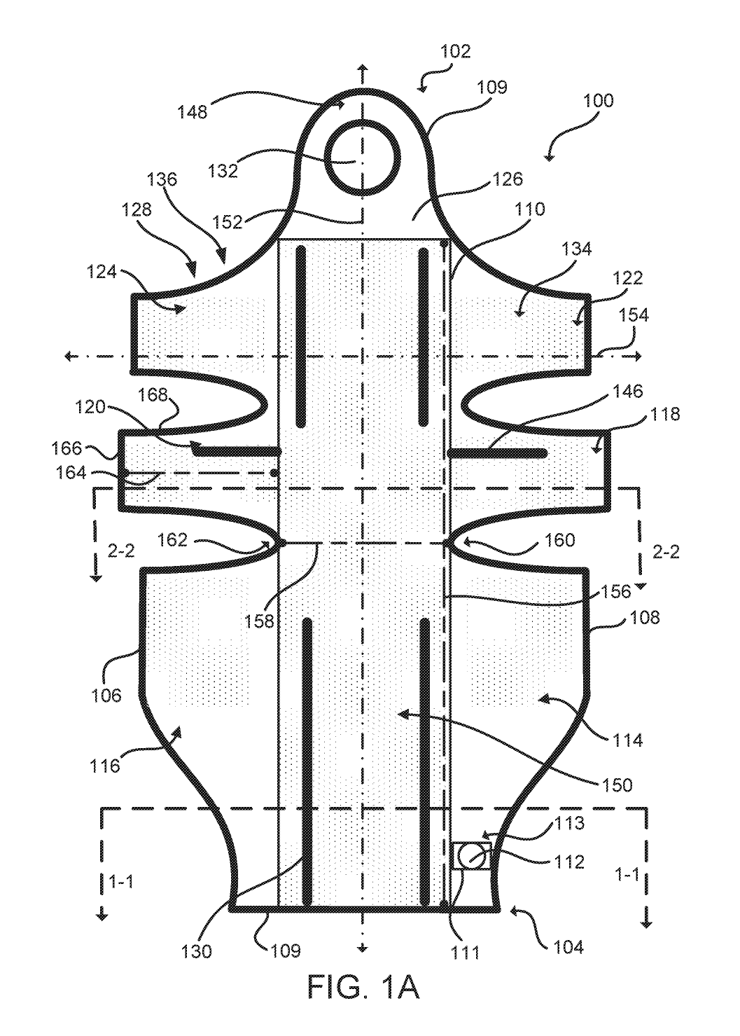

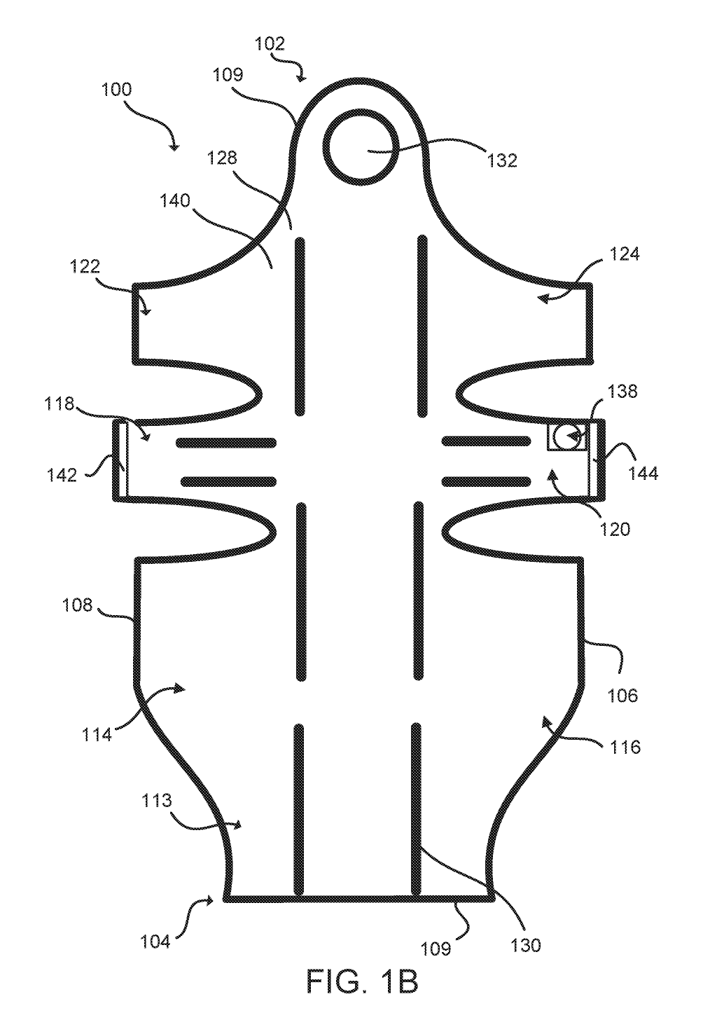

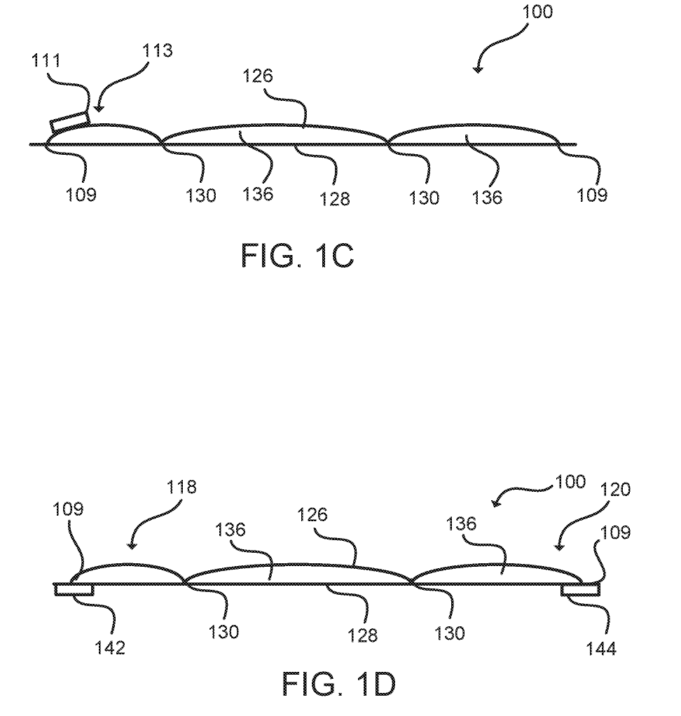

An underbody convective warming blanket 100 is seen in plan view in FIG. 1A, looking toward an upper side 110. The underbody blanket 100 has a head end 102, a foot end 104, and two elongate edges 106 and 108. The upper side 110 is generally permeable, with exceptions as may be required. The upper side 110 can have one or more holes 134 formed in the upper sheet 126 therein. The holes 134 are generally sized to allow air to flow to a person without deflation of the inflatable structure. Preferably, the holes 134 are no larger than 2 mm², or in some embodiments, no larger than 1 mm². In at least one embodiment, the upper sheet 126 can have one or more slits, or apertures formed therein to allow air to flow to a person without deflation of the inflatable structure.

The underbody blanket 100 may be constructed as described above from sheets (e.g., the upper sheet 126 and lower sheet 128) sealed together by a peripheral seal 109 extending proximate to the periphery of the underbody blanket 100.

At least one inlet port 113 is provided near an edge of the underbody blanket 100 to admit a stream of warmed pressurized air into the interior space 136 (creating an inflatable structure) of the underbody blanket 100. In at least one embodiment, the inflatable structure refers to an inflated state of the interior space 136 being filled with pressurized air. In at least one embodiment, the inlet port 113 is configured to secure a hose. The hose is configured to receive an air flow from an air source and direct the air flow into the interior space 136. For example, the at least one inlet port may be located at the edge 108 and may open through the peripheral seal 109. For example, one inlet port 113 is disposed near the foot end 104 of the underbody blanket 100. In some aspects, the inlet port 113 may be constituted of a collar (i.e., 111) having an opening 112 formed therein of relatively stiff material such as cardboard, that surrounds an opening into the interior space 136 of the underbody blanket 100. In this case, the opening 112 of collar 111 receives and retains the nozzle of an air hose while the air hose delivers a stream of warmed, pressurized air into the interior space 136. If not used, the collar 111 may be closed for example, by a removable plug or by an unpunctured portion of the upper sheet 126. In some aspects, the underbody blanket may be provided with two or more inlet ports which may be constructed and selectively operated with plugs as taught in the assignee's U.S. Pat. No. 5,997,572. Alternatively, the inlet ports 113 may be sleeves of material with means to retain the nozzle of an air hose.

As seen in FIG. 1A, the sheets of which the inflatable underbody convective blanket 100 is made are sealed together at line seals 130 within the peripheral seal 109 to form or define an air distribution structure that distributes warmed pressurized air in the interior space 136 of the underbody blanket. Some of these line seals 130 may include drain passages to draw off fluids from the surface 110 that may accumulate before, during and/or after surgery. Drain passages may be constructed as taught in the referenced U.S. Pat. No. 6,102,936, or in other configurations, as required. The line seals 130 are generally continuous and fluid impervious and are shown to be linear, i.e., substantially strait. However, the line seals 130 can take on a variety of shapes such as curves, circles, right angles, and so forth. The line seals 130 are formed through similar methods as used to form the peripheral seal 109 as discussed herein. Although line seals 130 are shown as being substantially parallel to the head end 102 and foot end 104 orientation, other seals can be used in various locations to improve airflow. For example, line seals 146 are horizontally disposed parallel to the wing portion to facilitate airflow into the distal end of a wing portion.

In at least one embodiment, the sheets are sealed together using a plurality of staked seals (which may be referred to as spot seals). The staked seals are generally circular but can be any geometric shape such as oval, triangular, rectangular, etc. Staked seals can differ from line seals in that line seals form a continuous line. Generally, a staked seal can bond an area of the upper sheet and the lower sheet. The area can be generally small. In at least one embodiment, the area is between 2 mm² to 4 cm², inclusive.

With further reference to FIG. 1A, in some aspects non-inflatable and/or impermeable areas 132 may be provided at the head end 102 of the underbody blanket 100. Warmed air cannot be convected from the areas 132, which are provided to receive and insulate the head of a person lying on the upper side 110 of the underbody blanket 100.

The blanket 100 can have a longitudinal axis. In at least one embodiment, the longitudinal axis 152 is defined by the head end 102 and foot end 104. In at least one embodiment, the longitudinal axis 152 is defined by an orientation of a person on the blanket 100. For example, longitudinal axis 152 can be formed by the intersection of the sagittal plane of the patient and the plane of the blanket 100.

The blanket 100 can also have a width axis. The width axis 154 can be defined by the edges 106 and 108. In at least one embodiment, the width axis 154 is defined by the orientation of the patient, wherein the width axis 154 is formed by the intersection of the transverse plane of the patient and the plane of the blanket.

The blanket 100 can have a head portion 148, a body portion 150, and at least one wing portion. The blanket 100 is shown with six wing portions, 114, 116, 118, 120, 122, and 124 for illustrative purposes only. In at least one embodiment, the blanket 100 can have at least four, at least six, or even at least eight wing portions.

The head portion 148 can be configured to receive a head of the patient. In at least one embodiment, the head portion 148 is similar in dimensions to a wing portion except that the head portion 148 generally extends distally along an axis parallel to the longitudinal axis. The head portion 148 may also be fluidically coupled to the body portion 150.

The body portion 150 can be configured to receive a posterior portion of a torso of the person. In at least one embodiment, the body portion 150 can be defined by a longitudinal dimension 156 and/or a width dimension 158. The body portion 150 can also have a longitudinal dimension 156 which can be the shortest dimension from one end of the peripheral seal 109 to another end of the peripheral seal 109 within the plane of the blanket 100. In at least one embodiment, the longest dimension can be measured along an axis parallel to the longitudinal axis 152. Similarly, a width dimension 158 of the body portion 150 is defined by a shortest dimension from one end of the peripheral seal 109 to another end of the peripheral seal 109 along an axis parallel to the width axis 154. In particular, the width dimension can be measured from at least one medial-intended portion (e.g., 160, 162). In at least one embodiment, the width and longitudinal dimensions are further characterized in that both dimensions are entirely within the peripheral seal 109. In at least one embodiment, the width and longitudinal dimensions are further characterized in that no portion of the longitudinal dimension intersects the peripheral seal 109.

Thus, the body portion 150 can be within the peripheral seal 109. In at least one embodiment, the body portion 150 (and the width dimension 158) can be at least partially defined by at least two medial-indented portions (i.e., the medial-most points of the peripheral seal) 160, 162 of the peripheral seal 109 measured along an axis parallel to the width axis 154 or perpendicular to the longitudinal axis 152. Although multiple medial-intended portions can exist within the blanket 100, the medial-most points (e.g., 160, 162) specifically refer to points that are closest to the sagittal plane of the patient.

A wing portion, or a flap can be a portion of the blanket 100 that does not contact the person in the down position but contacts the patient in the swaddled (i.e., folded over the patient) position. In at least one embodiment, part of the wing portion (e.g., the upper sheet) is configured to contact an anterior portion of the abdomen of the patient.

The wing portion can have a wing width dimension that is sufficient to allow the wing portion to intersect with the sagittal plane of the patient. In at least one embodiment, the wing dimension 164 can be measured from the body portion 150 to a distal end 166. Particularly, the wing dimension 164 can be measured along an axis parallel to the width axis 154. In at least one embodiment, the wing portion 120 can have a side 168 and a distal end 166. The side 168 and distal end 166 of the wing portion 120 can also be part of the peripheral seal 109. In at least one embodiment, the wing portion can be at least partially surrounded by the peripheral seal 109. For example, the peripheral seal 109 can form at least part of the border of the wing portion 120. In at least one embodiment, the wing width dimension 164 can be at least one-quarter of the width dimension 158. In at least one embodiment, the wing width dimension 164 can be a range from one-quarter of the width dimension 158 to one-half of the width dimension 158. While the wing width dimension 164 can be longer than one-half of the width dimension 158, further swaddling advantages may be lacking despite the extra material.

The wing portion length can be a measure of the distal end (e.g., 166). Generally, the wing portion length (e.g., 166) can be no greater than half of the longitudinal dimension 156.

The position of wing portion 120 can vary relative to other wing portions. For example, along the side 106, the wing portion 120 is spaced apart from wing portion 116 and wing portion 124. For example, the intersection of side 168 and distal end 166 has a special distance from a side of wing portion 124. In at least one embodiment, spaced apart can mean that the distal ends of each wing portion can be at least one eighth of the width dimension 158. For example, spaced apart can refer to a dimension measured along an axis parallel to the longitudinal axis. In at least one embodiment, wing portion 124 can be an opposing wing portion to wing portion 122. Opposing wing portions can be wing portions on opposite edges (e.g., 106, and 108).

Laterally, the positioning of the wing portions can vary depending on the configuration. In an inline configuration, an axis perpendicular to the longitudinal axis 152 can run through portions of both a wing portion (e.g., 120) from side 106 and an opposite wing portion on side 108 (e.g., 118).

FIG. 1B illustrates the underbody blanket 100 in plan view looking toward a lower side 140. Components of FIG. 1B, can be discussed further herein. The blanket 100 includes an inlet port 138 positioned on the lower side 140 and disposed on the lower sheet 128. In use, the wing portion 120 of the blanket 100 can drape over the abdomen of the person and allowing the air inlet port 138 to be inflated while the person is in the lying position.

Disposed on the wing portions 118, and 120 are attachment mechanisms 142 and 144. An attachment mechanism is any device that allows the secured attachment of one wing to another wing, preferably through a distal end. In at least one embodiment, the attachment mechanism is releasable. The attachment mechanism can be mechanical (e.g., mechanical fasteners such as hook-and-loop, buttons, sewing), or adhesive. In at least one embodiment, the attachment mechanism can be a string or rope-like device used to secure the section or a loop like attachment for an external strap or rope. In at least one embodiment, the attachment mechanism can be a malleable element (e.g., a wire, foil, and/or mesh) such that when a wing portion is folded over the patient (i.e., the swaddled position), the wing portion is self-forming. In at least one embodiment, self-forming means able to hold shape without having to be mechanically fastened to another component. The attachment mechanism 142 can be further configured to mate with attachment mechanism 144 (e.g., a button and loop).

FIGS. 1C-1D illustrate the interior space 136. Various components shown in FIGS. 1C-1D are described herein. The interior space 136 forms one or more internal air passageways sufficient to channel air from an air inlet through the upper sheet 126. A heater/blower unit and air hose may be used to provide a flow of warmed, pressurized air through the inlet port 113 into the interior space 136 of the underbody blanket 100.

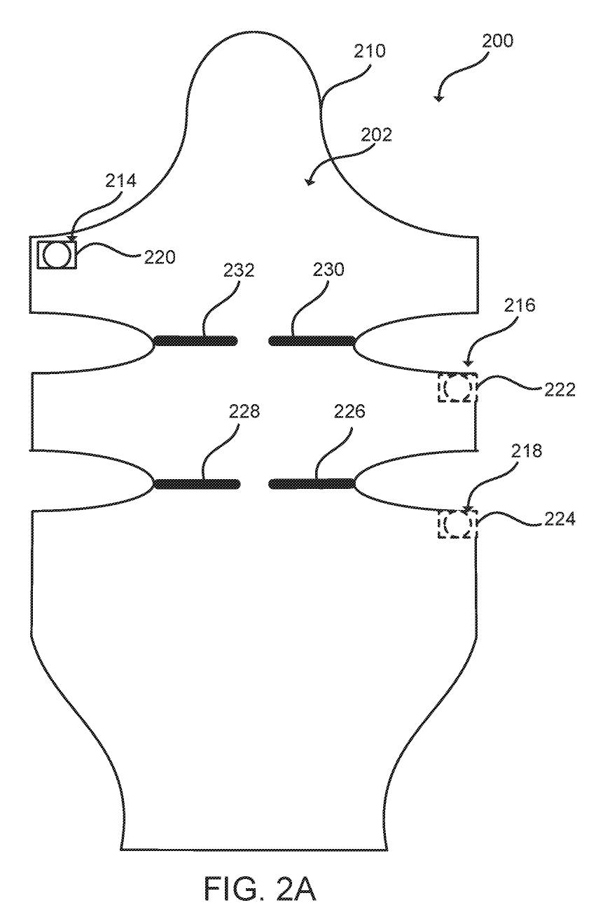

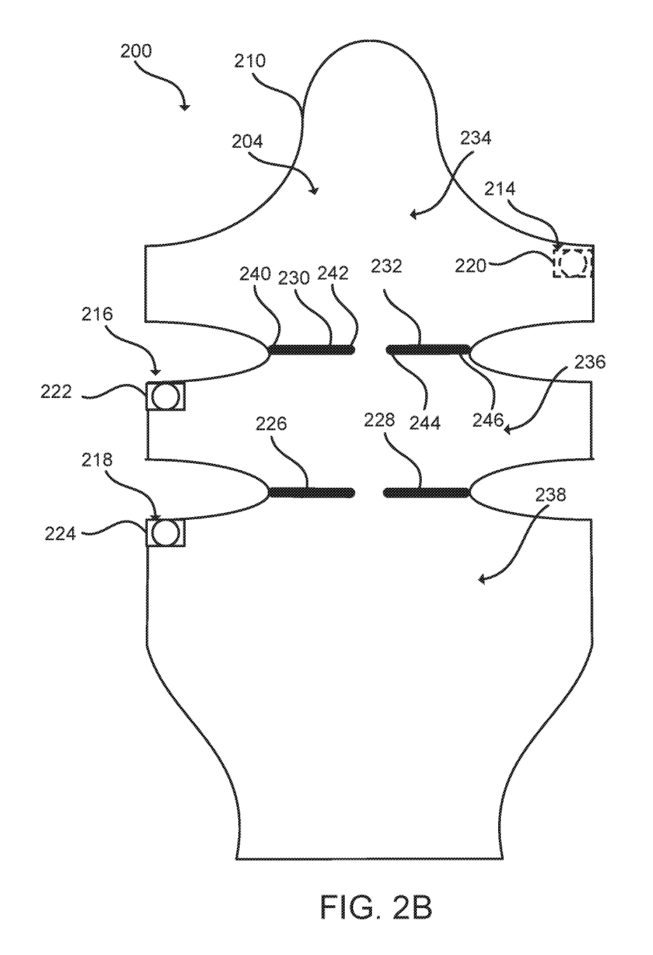

FIGS. 2A-2B illustrates an underbody blanket 200. The underbody blanket 200 has an upper sheet 202 and a lower sheet 204 bonded together with a peripheral seal 210, similar to the blanket 100 in FIG. 1. The blanket 200 has six wing portions, including wing portions 214, 216, and 218. Wing portion 214 can have an inlet port 220 disposed on the upper sheet 202 and inlet ports 222, 224 disposed on the lower sheet 204, specifically on wing portions 216, and 218. When the wing portions 216 and 218 are folded over a portion of the patient toward the medial plane of the patient (i.e., the swaddled position), then the inlet ports 220, 222, and 224 will be facing in toward one side. Thus, at least one inlet port disposed on a wing portion can be disposed on the opposite sheet from another inlet port.

In at least one embodiment, one or more line seals (e.g., 226, 228, 230, 232) may be present. The line seals can be similar to those described in blanket 100. As shown in FIGS. 2A-2B, the line seals can be positioned to isolate zones of heating. In at least one embodiment, at least two line seals can be used (e.g., 230, and 232) to create a zone. For example, line seals 230, 232 can be linear seals oriented parallel to the width axis (not shown). The line seals may function to restrict airflow into adjacent zones (e.g., from zone 234 to 236). The line seal 230 has a proximal end 240 (which is adjacent to or even contacts the peripheral seal 210) and a distal end 242. The line seal 232 has a proximal end 246 that is adjacent to (or contacts) the peripheral seal 210 and a distal end 244. In at least one embodiment, the distal end 242 is spaced apart from distal end 244. The distance between the distal ends 242, 244 is sufficient for air to be restricted between zones (e.g., between zone 234 and 236).

For example, the zone 234, 236, and 238 can each represent a zone of heating with different thermal properties. Zone 234 can be fed from inlet port 220, zone 236 can be supplied from inlet port 222, and zone 238 can be supplied from inlet port 224. The blanket 220 can further facilitate multiple air sources, thus having an advantage of using a smaller motor air source.

Further, certain zones can be cooler than other zones. For example, if air is supplied through only inlet port 222, then zone 236 may be warmer than zones 234 or 238.

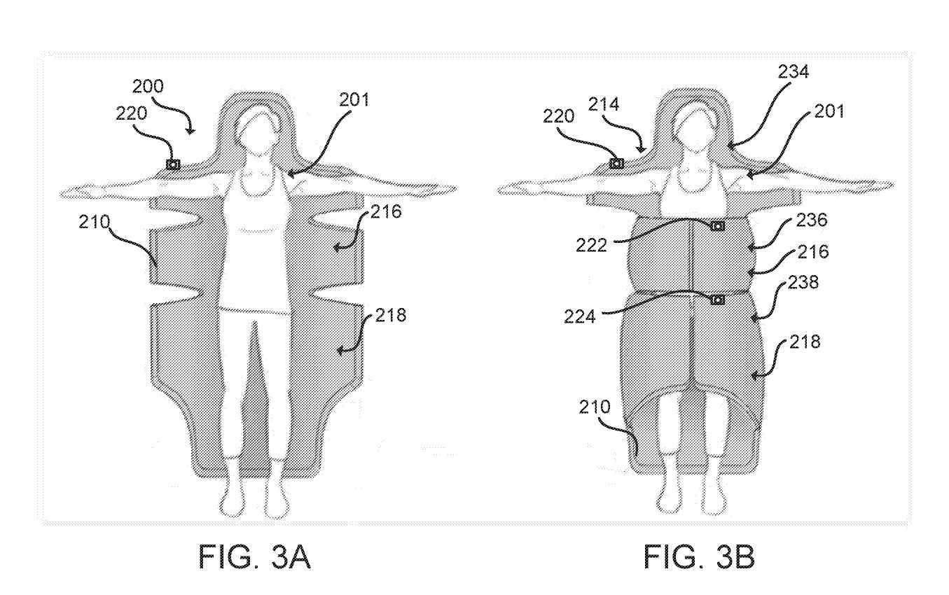

FIGS. 3A-3B illustrate the underbody blanket 200 with a patient 201 in both an unswaddled position (FIG. 3A) and swaddled position (FIG. 3B). Inlet ports 220, 222, 224 all face the same direction when the blanket 200 is in the swaddled position (FIG. 3B). In at least one embodiment, the swaddled position is achieved where a patient facing surface of a wing portion contacts an anterior portion of the patient 201. In at least one embodiment, the swaddled position of the blanket 200 can be achieved where a wing portion contacts another wing portion, preferably an opposing wing portion on the opposite edge. The swaddled position can further cause higher body temperatures to be retained at zones 236 and 238, whereas the wing portion 214 that is in the unswaddled position will likely cause a lower body temperature in zone 234. Various components, such as 210, 216, and 218 are described further herein.

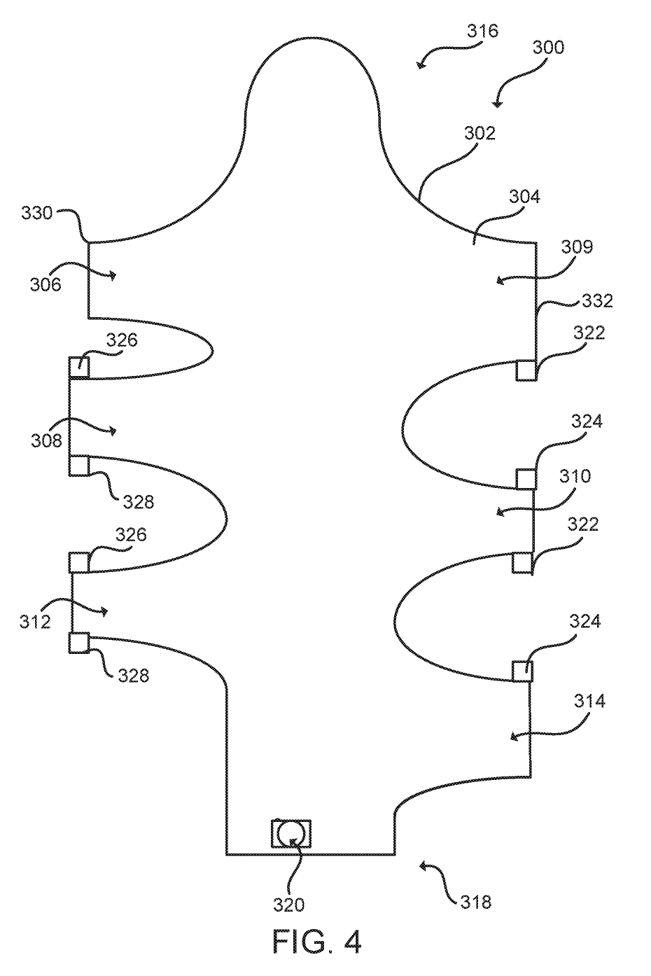

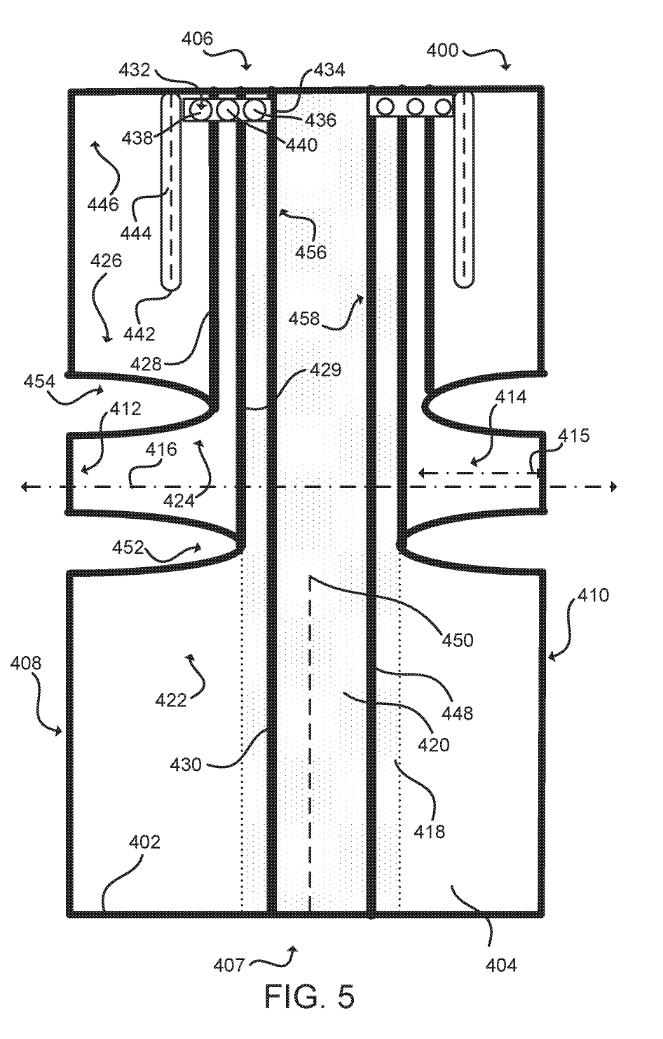

FIG. 4 illustrates an underbody blanket 300 having wing portions that interlace when the blanket 300 is in the swaddled position. Interlace can generally refer to one wing portion crossing together with another wing portion. The blanket 300 can be constructed similarly to blankets 100 and 200 described herein. For example, the blanket 300 can be formed from an upper sheet 304 and a lower sheet (not shown) bonded through a peripheral seal 302. The blanket 300 can have an inlet port 320 disposed on the upper sheet 304 and capable of being fluidically coupled to an interior space of the blanket 300.

The blanket 300 can have a head end 316 and a foot end 318, and a first elongate edge 330 and a second elongate edge 332. The blanket 300 can also have a plurality of wing portions. Wing portion 306 can be aligned with wing portion 308 meaning that an axis parallel to the width axis can intersect a portion of both wing portion 306 and wing portion 308.

In at least one embodiment, the wing portions are not aligned. For example, although blanket 300 is not drawn to scale, wing portion 308 is not aligned with wing portion 310 since an axis parallel to the width axis would not intersect with a portion of wing portion 308.

In at least one embodiment, the wing portions may have one or more attachment mechanisms (e.g. 322, 324, 326, 328). The attachment mechanisms may be disposed proximate or even adjacent to an intersection of a distal end with a side end of a wing portion. The attachment mechanisms can be configured to mate two opposing wing portions at a corner of the wing portion. For example, attachment mechanism 328 can mate with attachment mechanism 324 which releasably attaches wing portion 308 with wing portion 310. Further attachments are possible in similar manners with wing portion 310 being releasably attached to wing portion 312 and wing portion 312 being attached to wing portion 314. Thus, wing portions (e.g., 308, 312) proximate to the first elongate edge 330 can be interlaced with wing portions (e.g., 310, 314) proximate to the second elongate edge 332. In at least one embodiment, only the wing portions that are not aligned can be interlaced. Wing portions that are aligned (e.g., wing portions 309 and 306) cannot be interlaced.

FIG. 5 illustrates a blanket 400 having a plurality of ducts fluidically coupled to a plurality of wings. Blanket 400 can have a similar construction to blankets 100, 200, and 300 as described herein. For example, blanket 400 can be constructed using two sheets, an upper sheet 404 and a lower sheet (not shown) bonded proximate a periphery along a peripheral seal 402.

An aspect of blanket 400 is that the peripheral areas of a patient are warmed without warming the core of the patient, which can be useful during pre-warming of a patient.

The shape of the blanket 400 is generally rectangular. The blanket 400 can have a head end 406, a foot end 407, a first elongate edge 408, and a second elongate edge 410. The blanket 400 can have wing portions e.g., 412, and 414. In at least one embodiment, the wing portion can be positioned proximate to a horizontal midline 416 of the blanket 400. The midline 416 can divide the blanket 400 in half along a width axis. In at least one embodiment, the midline 416 intersects a portion of at least one of the wing portions 412. In at least one embodiment, the midline 415 of the wing portions is defined by a medial axis of the wing portion. The midline 415 can also be positioned from one-fourth of the longitudinal dimension to three-fourths of the longitudinal dimension measured from the head end 406.

In at least one embodiment, the plurality of ducts as described herein can be divided into a first set of ducts 456 and a second set of ducts 458. The first set of ducts are fluidically coupled to the wing portions (e.g., 412) that is proximate to the first elongate edge 408. The second set of ducts are fluidically coupled to a wing portion proximate to the second elongate edge 410. Both the first and second set of ducts have different inlet port manifolds (e.g., 432).

The first set of ducts 456 can comprise a plurality of line seals (428, 429, 430). For the purposes of clarity, only the first set of ducts 456 will be described in detail. The second set of ducts 458 can be structured similarly. In at least one embodiment, a portion of a duct may reside on the body portion 418. A duct as used herein can refer to an air passageway between an inlet port and an outside surface of a wing portion. The duct can also be referred to as an air chamber.

Duct 426 can be formed from the line seal 428. In particular, the line seal 428 can contact the peripheral seal 402 and a portion of the wing portion 412 that corresponds to the peripheral seal 402 or a medial-indented portion 454 of the peripheral seal 402. The duct 426 can receive air from the inlet port 432, particularly from hole 438. Duct 426 can receive air from an air source through hole 438 and apply the air toward the patient.

In at least one embodiment, duct 426 can have a non-inflatable area 442 with a line of weakness 444 disposed thereon. The non-inflatable area 442 can fluidically isolate the line of weakness 444 such that when torn, the blanket 400 does not leak air. The line of weakness 444 are generally perforations between the upper sheet 404 and a lower sheet. However, the line of weakness 444 can be any feature that allows for tearing of the blanket 400. As shown, when torn at the line of weakness 444, region 446 can be folded down toward the foot end or rolled toward the foot end, or even draped over a patient's arm to promote further heat retention.

The inlet port 432 can be from a collar 434 made from relatively stiff material such as cardstock, or a plastic card. The inlet port 432 can be configured to have a plurality of openings and thus resembles an inlet port manifold. The collar 434 can have a plurality of holes, e.g., 438, 440, 436, formed therein. In at least one embodiment, the upper sheet 404 may be present through the holes and the upper sheet 404 may be punctured to create a fluid pathway between an air source and an air permeable surface. An aspect of inlet port 432 is that each hole is isolated by a line seal such that each hole leads to a different duct. For example, hole 440 can fluidically couple to duct 424, hole 436 can fluidically couple to duct 422.

Duct 424 can be created through line seal 428 and line seal 429. In at least one embodiment, the line seal 428 can intersect with two proximate portions of the peripheral seal 402. In at least one embodiment, any line seal can intersect with a medial-indented portion (e.g., 452, 454) of the peripheral seal 402. Duct 422 can be formed through line seal 429 and line seal 430. In at least one embodiment, the line seal 430 and corresponding line seal 448 proximate the second elongated edge 410 can both fluidically isolate (thus forming a non-inflatable area 420) a portion of the body portion 418. For example, line seal 430 and the corresponding line seal 448 can intersect the peripheral seal 402 at opposing ends. A line of weakness 450 may also be present in the non-inflatable area 420. The line of weakness 450 can be a length that roughly corresponds to a patient inseam to partially wrap the legs of the patient.
List of illustrative embodiments:
1. A convective blanket comprising:
   an air permeable upper sheet having a periphery;
   a lower sheet bonded to the upper sheet with a peripheral seal proximate to the periphery of the upper sheet to form an interior space therein between the upper and lower sheets;
   a wing portion partially surrounded by a portion of the peripheral seal; and
   a body portion.
2. The convective blanket of any of the preceding Embodiments, wherein the body portion is configured to receive a posterior portion of a torso of a patient.
3. The convective blanket of any of the preceding Embodiments, wherein the wing portion is configured to contact an anterior portion of an abdomen of the patient when in a swaddled position.
4. The convective blanket of any of the preceding Embodiments, wherein the blanket has a head end and a foot end, wherein the wing portion is positioned between the head end and the foot end.
5. The convective blanket of any of the preceding Embodiments, wherein the wing portion has a wing dimension such that the wing portion intersects with a sagittal plane of the patient.
6. The convective blanket of any of the preceding Embodiments, wherein the blanket comprises a longitudinal axis defined by the head end and the foot end of the blanket.
7. The convective blanket of any of the preceding Embodiments, wherein the blanket comprises a longitudinal axis defined by an orientation of the patient, wherein longitudinal axis is formed by the intersection of a sagittal plane of the patient and the plane of the blanket.
8. The blanket of any of the preceding Embodiments, wherein a longitudinal dimension is a shortest dimension from one end of the peripheral seal to another end of the peripheral seal along an axis parallel to the longitudinal axis, wherein the shortest dimension does not intersect a portion of the peripheral seal.
9. The blanket of any of the preceding Embodiments, wherein the body portion is defined by a width dimension and the longitudinal dimension.
10. The blanket of any of the preceding Embodiments, wherein the blanket comprises a width axis defined by the orientation of the patient, wherein the width axis is formed by an intersection of a transverse plane of the patient and the plane of the blanket.
11. The blanket of any of the preceding Embodiments, wherein a width dimension of the body portion is defined by a shortest dimension from one end of the peripheral seal to another end of the peripheral seal along an axis parallel to the width axis.
12. The blanket of any of the preceding Embodiments, wherein the body portion is partially defined by at least two medial-most points of the peripheral seal measured along an axis parallel to the width axis.
13. The blanket of any of the preceding Embodiments, wherein the width dimension does not intersect with any portion of the peripheral seal.
14. The blanket of any of the preceding Embodiments, wherein the body portion is within the peripheral seal.
15. The blanket of any of the preceding Embodiments, wherein at least four wing portions are fluidically coupled to the body portion.
16. The blanket of any of the preceding Embodiments, wherein at least six wing portions are fluidically coupled to the body portion.
17. The blanket of any of the preceding Embodiments, wherein the wing portion comprises a distal end and at least one side.
18. The blanket of any of the preceding Embodiments, wherein the distal end extends from the body portion.
19. The blanket of any of the preceding Embodiments, wherein the distal end is parallel to the longitudinal axis.
20. The blanket of any of the preceding Embodiments, wherein the distal end, and at least one side of the wing portion are part of the peripheral seal.
21. The blanket of any of the preceding Embodiments, further comprising a first elongate edge and a second elongate edge, wherein an elongate edges form part of the peripheral seal.
22. The blanket of any of the preceding Embodiments, wherein a first wing portion, and a second wing portion are disposed adjacent the first elongate edge.
23. The blanket of any of the preceding Embodiments, wherein at least one side of the first wing portion is spaced apart from the at least one side of the second wing portion at the first elongate edge.
24. The blanket of any of the preceding Embodiments, wherein spaced apart means that the distal ends between each wing portion are at least one eighth of the width dimension of the body portion.
25. The blanket of any of the preceding Embodiments, wherein a third wing portion, and a fourth wing portion are disposed adjacent the second elongate edge.
26. The blanket of any of the preceding Embodiments, wherein a portion of the first wing portion and a portion of the third wing portion both intersect with an axis parallel to the width axis.
27. The blanket of any of the preceding Embodiments, wherein a portion the second wing portion and a portion of a fourth wing portion both intersect with an axis perpendicular to the longitudinal axis.
28. The blanket of any of the preceding Embodiments, wherein an axis parallel to the width axis does not intersect with both a portion the first wing portion and a portion of a third wing portion.
29. The blanket of any of the preceding Embodiments, wherein an axis perpendicular to the longitudinal axis does not intersect with both a portion the second wing portion and a portion of the third or fourth wing portion.
30. The blanket of any of the preceding Embodiments, wherein a portion of the first wing portion is configured to interweave between the third wing portion and the fourth wing portion.
31. The blanket of any of the preceding Embodiments, wherein the wing portion is defined by a wing dimension, at least one side, and a distal end.
32. The blanket of any of the preceding Embodiments, wherein the wing dimension is the distance from the distal end to the body portion measured along an axis parallel to the width axis.
33. The blanket of any of the preceding Embodiments, wherein the wing dimension is at least one-fourth of the width dimension.
34. The blanket of any of the preceding Embodiments, wherein the wing dimension is at least one-half of the width dimension.
35. The blanket of any of the preceding Embodiments, wherein a second wing dimension defines a wing portion.
36. The blanket of any of the preceding Embodiments, wherein the second wing dimension is a longest distance between two opposing sides of a wing portion.
37. The blanket of any of the preceding Embodiments, wherein the second wing dimension is no greater than half of the longitudinal dimension of the body portion.
38. The convective blanket of any of the preceding Embodiments, wherein the blanket comprises a head portion configured to receive a head of the patient.
39. The blanket of any of the preceding Embodiments, wherein the blanket further comprises: a head portion that is fluidically coupled to the body portion, wherein the head portion extends distally from the body portion along an axis parallel to the longitudinal axis.
40. The blanket of any of the preceding Embodiments, wherein a non-inflatable area is disposed proximate to the head portion.
41. The blanket of any of the preceding Embodiments, wherein a non-inflatable area is disposed adjacent to the head portion.
42. The blanket of any of the preceding Embodiments, wherein a line of weakness is disposed within a non-inflatable area.
43. The convective blanket of any of the preceding Embodiments, wherein the blanket comprises a non-inflatable area disposed therein.
44. The blanket of any of the preceding Embodiments, wherein at least one wing portion has an attachment mechanism disposed thereon.
45. The blanket of any of the preceding Embodiments, wherein the attachment mechanism is disposed on the lower sheet.
46. The blanket of any of the preceding Embodiments, wherein the attachment mechanism is disposed adjacent to an intersection of a distal end and at least one side of a wing portion.
47. The blanket of any of the preceding Embodiments, wherein some of the wing portions on a first elongate edge are configured to interlace with some wing portions on a second elongate edge.
48. The blanket of any of the preceding Embodiments, wherein the attachment mechanism is a mechanical fastener, or adhesive.
49. The blanket of any of the preceding Embodiments, wherein the attachment mechanism is a handle.
50. The blanket of any of the preceding Embodiments, wherein the upper sheet comprises a plurality of holes formed from the upper sheet and sufficient to allow air to flow to a patient without deflation of an inflatable structure.
51. The blanket of any of the preceding Embodiments, wherein the upper sheet has a temperature sensor disposed thereon.
52. The blanket of any of the preceding Embodiments, wherein the peripheral seal uses heat, adhesive, mechanical, or ultrasonic bonding between the upper sheet and the lower sheet.
53. The blanket of any of the preceding Embodiments, further comprising a plurality of staked or spot seals formed from bonding an area of the upper sheet and the lower sheet, wherein the area is between 2 mm² to 4 cm², inclusive.
54. The blanket of any of the preceding Embodiments, further comprising a plurality of line seals.
55. The blanket of any of the preceding Embodiments, wherein the line seals comprise linear seals and curved seals.
56. The blanket of any of the preceding Embodiments, wherein the linear seals are substantially linear seals between the upper sheet and the lower sheet.
57. The blanket of any of the preceding Embodiments, wherein at least one line seal is oriented parallel to the width axis.
58. The blanket of any of the preceding Embodiments, wherein a linear seal is proximate to at least one side of a wing portion.
59. The blanket of any of the preceding Embodiments, wherein a line seal is disposed within a portion of the wing portion sufficient to form a channel.
60. The blanket of Embodiment 57, wherein the inflatable structure comprises at least two line seals oriented parallel to the width axis.
61. The blanket of Embodiment 60, wherein a proximal end of a first line seal contacts the peripheral seal, a proximal end of a second line seal contacts the peripheral seal, and the distal ends of the first line seal and the second line seal are spaced apart sufficient for air to be restricted.
62. The blanket of Embodiment 61, wherein the distal ends of the first line seal and the second line seal contact a valve.
63. The blanket of Embodiment 62, wherein the valve is electromechanical and is configured to control airflow from one portion of the first wing portion to one portion of the second wing portion.
64. The blanket of any of the preceding Embodiments, wherein the inflatable structure further comprises at least one inlet port for retaining a hose, the hose is configured to receive an air flow from an air source and direct the air flow into the interior space.
65. The blanket of any of the preceding Embodiments, wherein the at least one inlet port comprises a collar with a hole or opening disposed therein such that a sheet of material is closed unless the sheet is punctured by a hose or portion thereof.
66. The blanket of any of the preceding Embodiments, further comprising a first inlet port disposed on the upper sheet and a second inlet port disposed on the lower sheet.
67. The blanket of any of the preceding Embodiments, further comprising a line of weakness extending from the foot end.
68. The blanket of any of the preceding Embodiments, wherein the line of weakness intersects the peripheral seal for a distance corresponding to a length of an inseam for a patient.
69. The blanket of any of the preceding Embodiments, further comprising a line of weakness extending from the foot end to no more than one-half of the longitudinal dimension of the blanket.
70. The blanket of any of the preceding Embodiments, further comprising a line of weakness extending from the head end.
71. The blanket of any of the preceding Embodiments, further comprising at least two lines of weakness extending from the head end oriented along an axis parallel to the longitudinal axis.
72. The blanket of any of the preceding Embodiments, wherein the line of weakness extends from the head end to a position corresponding to an upper shoulder of the patient.
73. The blanket of any of the preceding Embodiments, wherein the blanket is substantially rectangular.
74. The blanket of any of the preceding Embodiments, further comprising a plurality of ducts formed from a plurality of line seals.
75. The blanket of any of the preceding Embodiments, wherein the plurality of ducts are positioned such that a portion of the body portion is an impermeable area.
76. The blanket of any of the preceding Embodiments, wherein the plurality of ducts are positioned such that one duct inflates at least one wing portion.
77. The blanket of any of the preceding Embodiments, wherein the plurality of ducts are positioned such that one duct inflates only one wing portion.
78. The blanket of any of the preceding Embodiments, wherein some ducts from the plurality of ducts are positioned adjacent to one another.
79. The blanket of any of the preceding Embodiments, wherein the plurality of ducts comprise a first set of ducts and a second set of ducts.
80. The blanket of any of the preceding Embodiments, wherein the first set of ducts are fluidically coupled to a wing portion proximate to the first elongate edge.
81. The blanket of any of the preceding Embodiments, wherein the second set of ducts are fluidically coupled to a wing portion proximate to the second elongate edge.
82. The blanket of any of the preceding Embodiments, wherein the first set of ducts and second set of ducts have different inlet port manifolds.
83. The blanket of any of the preceding Embodiments, wherein an inlet port manifold comprises a collar with a plurality of holes formed therein.
84. The blanket of any of the preceding Embodiments, wherein each hole from the plurality of holes is aligned with a duct.
85. The blanket of any of the preceding Embodiments, wherein a duct is formed from at least one line seal intersecting with a portion of the peripheral seal.
86. The blanket of any of the preceding Embodiments, wherein a duct is formed from a first line seal intersecting with a first portion of the peripheral seal and a second line seal intersecting with a second portion of the peripheral seal.
87. The blanket of any of the preceding Embodiments, wherein a duct is formed from at least one line seal intersecting with a medial-indented portion of the peripheral seal.
88. The blanket of any of the preceding Embodiments, wherein the duct is an air chamber formed therein by at least one line seal and a portion of the peripheral seal.
89. The blanket of any of the preceding Embodiments, wherein the lower sheet is reinforced sufficient to support a 300 pound patient when secured at the head end and the foot end.
90. The blanket of any of the preceding Embodiments, wherein the lower sheet comprises a low-friction coating disposed on an outer side.
91. The blanket of any of the preceding Embodiments, wherein the upper sheet or lower sheet comprises a plurality of layers bonded together.
92. A system, comprising:
   the convective blanket of any of Embodiments 1 to 91; and
   a patient.
93. The system of Embodiment 92, further comprising:
   an air source configured to move warmed air; and
   a hose configured to attach to the convective blanket through an inlet port of the convective blanket.
94. A method, comprising:
   placing a patient on the convective blanket of any of Embodiments 1 to 92;
   attaching the air source to the convective blanket;
   activating an air source.
95. The method of Embodiment 94, further comprising:
   wrapping a wing portion around a patient such that the distal end of the wing portion intersects the sagittal plane of the patient.
96. The method of Embodiment 95, wherein wrapping the wing portion comprises, moving the distal end of the wing portion toward the sagittal plane of the patient.
97. The method of Embodiment 96, further comprising: attaching an attachment mechanism from a first wing portion to an attachment mechanism from a second wing portion.
98. The method of any of the preceding Embodiments, further comprising:
   rolling a wing portion to form a rolled wing portion;
   using an attachment mechanism to secure the rolled wing portion.

The novel tools and methods disclosed and illustrated herein may suitably be practiced in the absence of any element or step which is not specifically disclosed in the specification, illustrated in the drawings, and/or exemplified in the embodiments of this application. Moreover, although one or more inventions have been described with reference to one or more preferred embodiments, it should be understood that various modifications can be made without departing from the inventions of the description and figures. Accordingly, the inventive principles described and illustrated herein are limited only by the following claims.

What is claimed is:

1. A convective blanket comprising:
an air permeable upper sheet having a periphery;
a lower sheet bonded to the upper sheet with a peripheral seal proximate to the periphery of the upper sheet to form an interior space therein between the upper and lower sheets;
a wing portion partially surrounded by a portion of the peripheral seal; and
a body portion.

2. The convective blanket of claim 1, wherein the wing portion is configured to contact an anterior portion of an abdomen of the patient when in a swaddled position.

3. The convective blanket of claim 1, wherein the wing portion has a wing dimension such that the wing portion intersects with a sagittal plane of the patient.

4. The convective blanket of claim 1, wherein the blanket comprises a longitudinal axis defined by an orientation of the patient, wherein longitudinal axis is formed by the intersection of a sagittal plane of the patient and the plane of the blanket, wherein the blanket comprises a width axis defined by the orientation of the patient, wherein the width axis is formed by an intersection of a transverse plane of the patient and the plane of the blanket.

5. The blanket of claim 4, wherein a longitudinal dimension is a shortest dimension from one end of the peripheral seal to another end of the peripheral seal along an axis parallel to the longitudinal axis, wherein the shortest dimension does not intersect a portion of the peripheral seal.

6. The blanket of claims 4, wherein a width dimension of the body portion is defined by a shortest dimension from one end of the peripheral seal to another end of the peripheral seal along an axis parallel to the width axis.

7. The blanket of claim 6, wherein the body portion is defined by the width dimension and the longitudinal dimension.

8. The blanket of claim 1, wherein the wing portion comprises a distal end and at least one side, wherein at least one side of a first wing portion is spaced apart from the at least one side of a second wing portion at the first elongate edge, wherein spaced apart means that the distal ends between each wing portion are at least one eighth of the width dimension of the body portion.

9. The blanket of claim 8, wherein a third wing portion, and a fourth wing portion are disposed adjacent the second elongate edge.

10. The blanket of claim 9, wherein a portion of the first wing portion and a portion of the third wing portion both intersect with an axis parallel to the width axis.

11. The blanket of claim 9, wherein a portion of the first wing portion is configured to interweave between the third wing portion and the fourth wing portion.

12. The blanket of claim 1, wherein a line of weakness is disposed within a non-inflatable area of the blanket.

13. The blanket of claim 1, wherein at least one wing portion has an attachment mechanism disposed thereon.

14. The blanket of claim 1, further comprising a plurality of staked or spot seals formed from bonding an area of the upper sheet and the lower sheet, wherein the area is between 2 mm² to 4 cm², inclusive.

15. The blanket of claim 1, wherein a plurality of ducts are positioned such that one duct inflates at least one wing portion.

16. The blanket of claim 1, wherein the lower sheet is reinforced sufficient to support a 300 pound patient when secured at a head end and a foot end.

17. A system, comprising:
a convective blanket, comprising:
an air permeable upper sheet having a periphery;
a lower sheet bonded to the upper sheet with a peripheral seal proximate to the periphery of the upper sheet to form an interior space therein between the upper and lower sheets;
a wing portion partially surrounded by a portion of the peripheral seal; and
a body portion;
a patient.

18. The system of claim 17, further comprising:
an air source configured to move warmed air; and
a hose configured to attach to the convective blanket through an inlet port of the convective blanket.

19. A method, comprising:
placing a patient on the convective blanket, comprising:
an air permeable upper sheet having a periphery;
a lower sheet bonded to the upper sheet with a peripheral seal proximate to the periphery of the upper sheet to form an interior space therein between the upper and lower sheets;
a wing portion partially surrounded by a portion of the peripheral seal; and
a body portion;
attaching the air source to the convective blanket; and
activating an air source.

20. The method of claim 19, further comprising:
wrapping a wing portion around a patient such that the distal end of the wing portion intersects the sagittal plane of the patient.
